SEQUENCE LISTING (1 sequence)

SEQ ID NO: 1 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

(19) United States
(12) Patent Application Publication
(10) Pub. No.: US 2016/0327559 A1
(43) Pub. Date: Nov. 10, 2016

(54) HEPATOCYTE GROWTH FACTOR AS MARKER OF PROGNOSIS IN SMALL CELL LUNG CANCER (SCLC)

(71) Applicant: FUNDACIÓ INSTITUT MAR D'INVESTIGACIONS MÈDIQUES (IMIM), Barcelona (ES)

(72) Inventors: Edurne ARRIOLA APERRIBAY, Barcelona (ES); Israel CAÑADAS CASTILLO, Barcelona (ES); Ana ROVIRA GUERIN, Barcelona (ES); Joan ALBANELL MESTRES, Barcelona (ES)

(21) Appl. No.: 15/108,249

(22) PCT Filed: Apr. 24, 2014
(86) PCT No.: PCT/EP2014/058375

§ 371 Date: Jun. 24, 2016

(51) Int. Cl. G01N 33/574

(52) U.S. Cl. CPC G01N 33/57423; G01N 2333/4753; G01N 2800/52

(57) ABSTRACT

The invention relates to a new marker, the hepatocyte growth factor (HGF) in serum or plasma for the prognosis of small-cell lung cancer (SCLC). The invention also provides methods for selecting subjects suffering from SCLC that are candidate to respond to certain substances inhibiting certain kinases expressed in the tumours. There are also encompassed the use of immunoassay and nucleic acid reagents for detecting HGF in serum or plasma, said reagents for carrying out the prognostic method.

### FIELD OF THE INVENTION

The present invention relates to the field of medicine, in particular to the field of cancer diagnosis and prognosis, in particular to the ultimate aim of selecting an appropriate therapy. It provides methods for the diagnosis and/or prognosis of cancers, namely of lung cancers.

### BACKGROUND ART

Small-cell lung cancer (SCLC), also known as Small-cell carcinoma or “oat-cell carcinoma”, is a highly malignant cancer that most commonly arises within the lung, although it can occasionally arise in other body sites, such as the cervix, prostate, and gastrointestinal tract.

SCLC is a highly lethal disease and accounts for approximately 15% of patients with lung cancers. Although many genetic alterations have been identified with potential therapeutic interest, no targeted treatment has been successful to date in improving the outcome of patients. Outcome in advance stage remains poor with a median overall survival that does not exceed one year with available treatments. The research of novel targets and treatments for selected patient populations in this disease is therefore urgently needed.

Moreover, correlative biomarker studies for selecting a therapy, as well as markers for the evaluation of the prognosis of the disease are still lacking. Determining prognosis may be useful in order to further select an appropriate therapy regimen or a palliative one.

Nowadays the diagnosis and prognosis of SCLC is performed basically by determining physiological symptoms, such as a persistent cough that goes worse; chest pain with deep breathing; hoarseness; weight loss or loss of appetite; spits or phlegm with blood or colored, etc. These listing of physiological symptoms are completed with imaging methodologies of lungs (X-Ray, positron emission tomography (PET), Computed Tomography scan (CT), and Magnetic Resonance Imaging (MRI)). Finally, the analysis of the lung cells is performed. These cells may come from sputum or phlegm (spitum citology), from fluid removed around the lung, or from a biopsy of a suspicious area.

Some cell markers have been elucidated for the prognosis or evaluation of the severity of the disease. At this respect, it has been reported that activation of the transmembrane receptor tyrosine kinase known as MET is overexpressed in many solid tumours with poor outcome (poor prognosis). In addition, a part of overexpression, activation of MET by phosphorylation, that is, detection of the expression of phosphorylated MET receptor tyrosine kinase (p-MET) is associated with decreased survival in SCLC, as depicted by Arriola et al, “MET phosphorylation predicts poor outcome in small cell lung carcinoma and its inhibition blocks HGF-induced effects in MET mutant cell lines”, Br J Cancer—2011, Vol—No. 105(6), pp.: 814-23. Human tyrosine kinase Met corresponds to the entry sequence in UniProtKB database with Accession Number P08581, sequence Version 4 of Jul. 7, 2009 (retrieved from version 193 of the database, last modified on Mar. 19, 2014)

Also the document of Cañadas et al., “Targeting epithelial to mesenchymal transition with Met inhibitors reverts chemoresistance in small cell lung cancer”, Clin Cancer Res—2013, (http://clincancerres.aacrjournals.org/lookup/doi/10.1158/1078-0432.CCR-13-1330) shows in preclinical SCLC models that hepatocyte growth factor (HGF) induces epithelial to mesenchymal transition (EMT) that results in increased tumourogenesis, invasiveness and chemoresistance. The document also reports an association between MET activation and mesenchymal markers (vimentin, Snail1, SPARC) in human SCLC samples, this association being correlated with a poor outcome. Furthermore, mesenchymal markers were upregulated in relapsed, chemorefractory disease. These data provide rational to consider clinical trials combining chemotherapy with MET inhibitors in SCLC patients with a mesenchymal/MET activated phenotype. Nonetheless, the detection of mesenchymal markers is to be performed in biopsies, which is always an invasive not always feasible sample mode.

All the data retrieved by Arriola et al. and Cañadas et al. are evaluated in cells, which means the use of biopsies of the lung tissue. Biopsies are prepared to carry out immunohistochemistry analysis for detecting the markers. One of the problems or limitations associated to immunohistochemistry techniques proceeds from the need of establishing an arbitrary cut-off to classify the patient in a particular stage of the disease and, if needed, to select the more accurate treatment. Since immunohistochemistry analysis is not a quantification method but a “visual” method, many times this technique is deficient in providing accuracy to differentiate between two stages of the disease.

Therefore, new specific biomarkers for the diagnosis and prognosis of SCLC are needed. In particular, markers for the analysis of the prognosis of SCLC, which may aid to select an appropriate treatment. More in particular, there is the need of predictive biomarkers for MET therapies.

### SUMMARY OF THE INVENTION

Inventors determined that serum and plasma levels of human hepatocyte growth factor (HGF) were predicting, in an independently manner, the outcome in patients with Small Cell Lung Cancer (SCLC). Patients with high serum hepatocyte growth factor (herewith abbreviated sHGF) showed clearly shortened survival and an incremental risk for death was found with increasing levels of sHGF at the moment of diagnosis, said diagnosis performed previously with other means.

Thus it is a first aspect of the invention an in vitro method for the prognosis of small cell lung cancer (SCLC) which comprises the step of determining in an isolated sample of a subject the level of hepatocyte growth factor (HGF), wherein the sample is selected from serum and plasma.

This aspect may also be formulated as an in vitro method for predicting the outcome in SCLC diagnosed subjects, the method comprising determining the serum or plasma HGF levels or amounts in an isolated sample. The prediction of the outcome is to be understood as the prognosis of the disease.

Human HGF is generally secreted by mesenchymal cells in the stroma of the tumors and in some case, such as in SCLC, it can be secreted by tumour cells. It targets and acts primarily upon epithelial cells and endothelial cells, but also acts on haemopoietic progenitor cells. HGF regulates cell growth, cell motility, and morphogenesis by activating the tyrosine kinase signaling cascade/pathway after binding to the proto-oncogenic c-Met receptor (MET). Its ability to stimulate mitogenesis, cell motility, and matrix invasion gives it a central role in angiogenesis, tumourogenesis, and tissue regeneration. Human HGF is codified in chromosome 7 and it is produced as a protein of 728 AA in its isoform 1, and has the amino acid sequence as disclosed in the Protein Knowledgebase (UniProtKB database) entry with Accession Number P14210 of Aug. 1, 1991, Version 2 (retrieved from version 179 of the database, last modified on Mar. 19, 2014). This isoform 1 is also disclosed in the present invention as SEQ ID NO: 1. There have been identified five additional isoforms, which result from alternative splicing of the mRNA identified in the GenBank database as NM_000601.4 (Version 4) of 2820 base pairs. Isoforms 2 to 6 correspond, respectively, to the Accession Numbers of the UniProtKB database P14210-2, P14210-3, P14210-4, P14210-5, and P14210-6 (retrieved from the same database version as isoform 1)

HGF is secreted by the cells as a single inactive polypeptide and is cleaved by serine proteases into a 69-kDa alpha-chain and 34-kDa beta-chain. A disulfide bond between the alpha and beta chains produces the active, heterodimeric molecule.

Serum levels of HGF, that is serum HGF (sHGF) have been associated with prognosis in several tumour, such as prognostic for stage II or III colorectal cancer (Toiyama et al., “Serum hepatocyte growth factor as a prognostic marker for stage II or III colorectal cancer patients”, Int J Cancer—2009, Vol. No. 125(7), pp.: 1657-62). Also it has been associated with early metastatic disease in primary lung cancer patients (Hosoda et al., “Plasma hepatocyte growth factor elevation may be associated with early metastatic disease in primary lung cancer patients”, Ann Thorac Cardiovasc Surg—2012, Vol. No. 18(1), pp.: 1-7). sHGF is also a prognostic marker for non-small cell lung cancer (Ujiie et al. “Serum hepatocyte growth factor and interleukin-6 are effective prognostic markers for non-small cell lung cancer”, Anticancer Res—2012, Vol. No. 32(8), pp.: 3251-8). However it has never been associated with the prognosis of SCLC.

It is noteworthy that inventors have also determined that sHGF levels are associated with a MET activation phenotype of the tumour. That is, sHGF levels correlate with an activated MET pathway, or which is the same with a. mesenchymal phenotype of the tumour cells (EMT). The inventors propose that this correlation of sHGF levels with the cell phenotype demonstrate also a link between the levels of this MET ligand (HGF) in the serum and the biological effects in the tumour. These results provide thus novel evidence of the biological relevance of circulating HGF and are added to previous evidence supporting the potential role of MET inhibition in this lethal disease. Thus, determining in serum the amounts or levels of HGF represents an alternative way of detecting if the MET pathway is activated in a subject, previously diagnosed of SCLC, without the need of carrying out unpleasant biopsies.

More important is the fact that serum detection of HGF over a reference control value allows selecting patients to be treated with MET inhibitors.

Therefore, is also part of the invention an in vitro method for selecting a subject suffering from SCLC for a therapy regimen comprising MET pathway inhibitors, which method comprises the step of determining in an isolated sample of the subject the level of HGF, said sample being selected from serum and plasma.

This aspect can also be formulated as a method for selecting a therapy for a patient having SCLC, comprising determining in an isolated sample of the subject the level of HGF, said sample being selected from serum and plasma; and selecting, based on the level of HGF, a therapy regimen that includes the administration of MET pathway inhibitors. The invention encompasses a method for treating a patient suffering from SCLC, comprising determining in an isolated sample of the subject the level of HGF, said sample being selected from serum and plasma; and treating the patient, based on the amount of HGF, with a therapy regimen comprising MET pathway inhibitors.

Subjects selected or recommended for a therapy regimen comprising MET pathway inhibitors may further be recommended for chemotherapy, since inhibition of MET pathway in SCLC subjects re-sensitizes cells to chemotherapy (Cañadas et al., supra).

Hence, the invention provides a way for detecting a subpopulation of subjects suffering from SCLC that show activated MET pathway and a mesenchymal phenotype of the tumour. Detection of high levels of serum or plasma HGF provides thus information of an aggressive phenotype of the tumour, usually resistant to chemotherapy but sensitive to MET pathway inhibitors. As a whole, all these data allow deciding or recommending an effective therapy regimen avoiding costs and saving time, being the time of special value in this cancer type.

Although plasma levels of HGF are different from those detected in serums, as will be depicted below, plasma levels of HGF (pHGF) also correlate with the disease and, thus, the skilled man will know the levels or amounts in plasma that are indicative of SCLC and, in particular, of the prognosis of the disease.

Interestingly too, it is the fact that sHGF levels at diagnosis of the disease by other means and changes during treatment have a real impact in the prognosis of the disease. Thus, yet another aspect of the invention is an in vitro method for determining the overall survival of SCLC in a subject diagnosed of this disease and receiving a therapy regimen, comprising determining the level of hepatocyte growing factor (HGF) in isolated serum or plasma samples of the subject at different times along survival of the subject and comparing said levels, being the overall survival defined as the survival time (months, years) from diagnosis of SCLC to death for any cause

All these methods provide evidences of the new and advantageous use of serum or plasma HGF as biomarker for the prognosis of SCLC. In particular, all these methods stem from the fact that serum or plasma levels (amounts) of HGF are useful to assess the malignancy or stage of the tumour in a subject.

Another aspect of the invention include the use of immunoassay reagents for determining the level of serum or plasma hepatocyte growth factor, for carrying out the methods as defined above, that is, for assessing the prognosis of SCLC in terms for example of overall survival of the disease, and/or for selecting a therapy regimen for a subject suffering from SCLC, or for selecting a SCLC suffering subject for being recommended to receive a particular therapy regimen. These immunoassay reagents may be forming part of kits also for the same purpose (i.e. detecting in a sample the presence and amounts/levels of serum or plasma HGF). This aspect can also be formulated as a method for the prognosis of SCLC, a method for selecting a therapy regimen comprising MET pathway inhibitors and a method for determining the overall survival of a subject suffering from SCLC, wherein the method comprises determining the level of serum or plasma hepatocyte growth factor with immunoassay reagents.

In the same, way another aspect is the use of nucleic acid analysis reagents for determining the level of serum or plasma hepatocyte growth factor, for carrying out the methods as defined above. These reagents may also form part of a kit. This aspect can also be formulated as a method for the prognosis of SCLC, a method for selecting a therapy regimen comprising MET pathway inhibitors and a method for determining the overall survival of a subject suffering from SCLC, wherein the method comprises determining the level of serum or plasma hepatocyte growth factor with nucleic acid reagents.

As will be illustrated in the examples below, the methods of the invention provide for the first time a way for determining the prognosis of SCLC in isolated serum and plasma samples of subjects diagnosed previously of SCLC, said methods imply the advantage of fastly providing the relevant information for selecting a therapy elongating subject overall survival. In addition, the methods serve for the follow-up of subjects diagnosed of SCLC for determining prognosis and evolution of the disease after treatment (if applied). All these information are useful in taking appropriate decisions by the professional side, which in turn can inform the patient, both concealing a way of facing the disease.

### BRIEF DESCRIPTION OF THE DRAWINGS

FIG. 1, relating to Example 1 is a graph with the distribution of the baseline (before treatment) levels of serum HGF in the Y-axis (B sHGF in pg/ml) determined in a cohort of healthy control subjects (H, N=30) and in subjects suffering from SCLC (N=104). Median values are indicated by the horizontal line in the graphics. The standard deviation is indicated by the vertical lines.

FIG. 2, relating to Example 2, is a Kaplan-Meyer graphic or curve. It relates to Example 2 and shows the Overall survival (OS) of SCLC patients having sHGF levels higher than 1886 pg/ml (dashed line) and of patient with sHGF levels lower that 1886 pg/ml (continuous line). In these type of graphics, the X-axis shows time in months (T(m)).

FIG. 3, relating to Example 2, is another Kaplan-Meyer curve, in this case showing the Overall survival (OS, Y-axis) along time in months (T(m), X-axis) of SCLC patients. SCLC patients that showed a decrease of the sHGF levels from baseline (B) to response evaluation after treatment (R) are indicated in continuous line. SCLC patients that showed an increase of the sHGF levels from baseline (B) to response evaluation after treatment (R) are indicated in dashed line.

FIG. 4, relating also to Example 2, is a Kaplan-Meyer curve showing the Overall survival (OS, Y-axis) along time in months (T(m), X-axis) of SCLC patients. SCLC patients at Stage IV, which showed a decrease of the sHGF levels from baseline (B) to progression (P), are indicated in continuous line. SCLC patients, which showed an increase of the sHGF levels from baseline (B) to progression (P), are indicated in dashed line.

### DETAILED DESCRIPTION OF THE INVENTION

The following definitions are provided for the purpose of understanding and for making easy the comprehension of the invention.

In the sense of the present invention the terms “amount of HGF” or “level of HGF” are used interchangeably as synonymous concepts and relate to the concentration (mass per volume unit of sample) of the HGF. Serum and plasma levels of HGF are also abbreviated as sHGF and pHGF. When in the present invention it is indicated that serum or plasma HGF is determined in an isolated sample, is to be understood that any of the isoforms 1 to 6 of the protein is detected, or a combination of these isoforms.

The expression “Reference control value” or “reference control level” (used herewith interchangeably) is to be understood as the level/amount of HGF from which a particular correlation with the disease (i.e. SCLC) is performed. Generally, it is the serum or plasma median amount of HGF resulting of samples from a cohort of subjects, in the present case subjects suffering from SCLC. The median is defined as the numerical value separating the higher half of a data sample, a population, or a probability distribution, from the lower half. The samples may be taken from a subject or group of subjects wherein the presence, absence, stage, or course of the disease has been properly performed previously. This value is used as a threshold to discriminate subjects wherein the condition to be analyzed is present from those wherein such condition is absent. Reference control values are usually determined considering similar characteristics of the subjects (age, sex, race, etc.). Nonetheless, a reference control value may also be defined to locate a value discriminating within some particular features among a type of subject suffering from SCLC and another type also suffering from the disease. In addition, the reference control value may be a value from the same subject but measured at different time points. In addition, the reference control level may be determined based on the levels of HGF in serum or plasma detected before any kind of treatment in a patient of cancer, or a population of the patients whose disease state (good or poor prognosis) is known. The skilled person in the art, making use of the general knowledge, is able to choose the subject or group of subjects more adequate for obtaining the reference control level for each of the methods of the present invention. Methods for obtaining the reference value from the group of subjects selected are well-known in the state of the art.

“Overall survival” or “Cumulative survival” (used interchangeably herewith) is to be understood as overall the survival time (months, years) from diagnosis of SCLC to death for any cause. In general terms, patients with a certain disease (for example, colorectal cancer) can die directly from that disease or from an unrelated cause (for example, a car accident). When the precise cause of death is not specified, this is called the overall survival rate or observed survival rate. Doctors often use mean overall survival rates to estimate the patient's prognosis. This is often expressed over standard time periods, like one, five, and ten years. The “survival rate” is a part of survival analysis, indicating the percentage of people in a study or treatment group who are alive for a given period of time after diagnosis. Survival rates are important for prognosis, but because this rate is based on the population as a whole, an individual prognosis may be different depending on newer treatments since the last statistical analysis as well as the overall general health of the patient The “Hazard ratio” abbreviated (HR) is the ratio of the hazard rates corresponding to the conditions described by two levels of an explanatory variable. It is also defined as the risk of suffering an event between two separate groups. A HR of 1.29 for SCLC indicates that these subjects suffering from SCL have 29% more probabilities of dying that those patients with a lower HR.

“Clinical progression” or “progression” means in the sense of the invention that a particular new detectable event has taken place throughout the evolution of the disease. In this particular case of SCLC a progression could be the growing of the tumour, metastasis or new lesions. Data in the present invention referring to progression correspond to the levels of HGF from isolated blood detected at one progression (generally the first after diagnosis). The invention provides for the first time serum and plasma HGF as prognosis biomarker of SCLC, a severe form of lung cancer in terms of short overall survival and high hazard ratios.

The expression “therapeutic regimen” is to be understood as encompassing either pharmacological therapies (such chemotherapy, administration of MET pathway inhibitors) as well as therapeutically surgical interventions, as well as other clinical decisions taken by the oncologist concerning, for instance, hospitalization or discharge decisions, or dietary or social habits pointed by the doctor such as salt ingestion, liquid intake or physical activity. For “therapy regimen comprising MET pathway inhibitors” is to be understood a therapy regimen wherein the subject receives drugs or compounds that inhibit the MET pathway, either by directly inhibiting the MET tyrosine kinase receptor, or drugs or compounds that block, inhibit or reduce the effect or action of a downstream protein of the MET signaling pathway. Examples of inhibitors that directly inhibit the MET tyrosine kinase receptor include anti-MET antibodies and kinase inhibitors, the later avoiding prevent ATP binding to MET, thus inhibiting receptor transphosphorylation and recruitment of the downstream effectors or proteins.

According to the present invention, it was newly discovered that serum or plasma HGF levels are significantly associated with poorer prognosis of patients suffering from SCLC. Thus, the present invention provides a method for determining or assessing the prognosis of a patient with cancer, in particular SCLC, by detecting the serum or plasma levels of the HGF in a isolated sample of the patient.

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower overall survival and post-treatment overall survival or by a higher hazard ratio. Conversely, a positive, favorable, or good prognosis is defined by an elevated overall survival or post-treatment overall survival.

The term “assessing the prognosis” refers to the ability of predicting, forecasting or correlating a given detection or measurement with a future outcome of cancer of the patient (e.g., malignancy, likelihood of curing cancer, survival, and the like). For example, a determination of the serum or plasma levels of HGF over time enables a predicting of an outcome for the patient (e.g., increase or decrease in malignancy, increase or decrease in grade of a cancer, likelihood of curing cancer, survival, and the like).

The in vitro method for the prognosis of small cell lung cancer (SCLC) comprises the step of determining in an isolated sample of a subject the levels of serum or plasma HGF. In a particular example of the method, the level of HGF is equal or higher than a reference control value, and the subject is determined as poor prognosis of SCLC. Thus, detection of higher amounts than said reference control value is not only confirming diagnosis previously done by other means, but also indicates the poor prognosis (outcome) of the disease. As above indicated, for poor prognosis is to be understood a poor outcome and evolution of the disease. In a particular embodiment, said reference control value may be the median of the levels of a cohort of SCLC subjects with a poor outcome (poor prognosis). Then, levels of HGF equal or higher than this reference control value defined by subjects of poor prognosis will be indicative of a poor prognosis for any test subject.

As above exposed, any reference control value may in addition depend on sex, age and population type, among other variables. Nonetheless, specific ranges including this variability will be determined for each patient type, so that the test sample is compared with the reference control value having more variables in common. The same reasoning is applicable when reference values are taken from comparison of two groups of subjects all suffering from SCLC but with some particular differences (i.e. patients with reduced levels after treatment vs patients with increased levels after treatment). The reference control value may also encompass the values of subjects suffering from the disease but at early stages of the disease, since it had been determined that the levels or amounts of HGF tend to increase along time.

In a particular embodiment of the in vitro prognosis method of the invention, if the level of serum HGF (sHGF) is equal or higher than 1500 pg/ml a poor prognosis of SCLC is determined.

In another particular embodiment, optionally in combination with any of the embodiments above or below, the level of sHGF is equal or higher than 1800 pg/ml. Inventors determined that a cut-off of 1886 pg/ml of HGF measured in serum allows discriminating SCLC patients of worse (poor) prognostic and short overall survival. Thus, if the level of sHGF is equal or higher than 1886 pg/ml a poor prognosis is determined. This cut-off was in particular determined from the median of the sHGF levels of a cohort of subject suffering from SCLC.

The prognosis of SCLC, defined as the medical term for predicting the likely outcome of subject's current standing, may be in particular evaluated or determined in the present case using the following parameters: the overall survival and the hazard ratio.

In general, the detection of high levels or amounts of serum or plasma HGF is indicative of poor prognosis of SCLC, since subjects with high levels have a short overall survival (OS) in respect of the subjects with SCLC not having these higher levels of the marker. With regard to the hazard ratio, the inventors determined, moreover, that increases in 1000 pg/ml of serum HGF in relation to previous measures were associated with a hazard ratio (HR) of 1.28 (1.14-1.42) (p<0.001) of dying. Thus, the probability of dying after an increase of this magnitude is 28% higher than before.

As above exposed, detection of these high levels of serum and plasma HGF in SCLC patients reflects the phenotype of the tumour. That is, there is a significant association between increased baseline levels of sHGF (above the median of a reference control value, in particular the median of a cohort of subjects suffering from SCLC) and the biopsy markers indicating an endothelial to mesenchymal transition (EMT) phenotype. It is known that markers for EMT are indicative of poor prognosis of SCLC but, at the same time, that these tumours may be faced with MET pathway inhibitors in order to re-sensitize the cells to chemotherapy. Thus, a subject suffering from SCLC can be selected for a therapy regimen comprising MET pathway inhibitors if serum or plasma HGF are determined and concluded that they correlate with markers indicating an endothelial to mesenchymal transition (EMT) phenotype.

In a particular embodiment of the in vitro method for selecting a subject suffering from SCLC for a therapy regimen comprising MET pathway inhibitors, the MET pathway inhibitors are selected from the group consisting of foretinib, crizotinib, onartuzumab, LY2875358, LY2801653, AMG-208, AMG-337, MGCD265, cabozantinib, golvatinib, rilotumumab, flicatuzumab, nintedanib, bevacizumab, dovitinib, danusertib, ponatinib, AZD4547, PD173074, and combinations thereof.

In a particular embodiment of the method for selecting a subject for a therapy regimen, the method comprising determining the levels of serum or plasma HGF in an isolated sample, if the level of HGF is equal or higher than a reference control value, a therapy regimen comprising MET pathway inhibitors is recommended. In a particular embodiment, optionally in combination with all the embodiments allow or below, the reference control value is the serum or plasma level of HGF resulting from the median of the levels of HGF of a cohort of subjects suffering from SCLC.

In another particular embodiment, if the level of serum HGF (sHGF) is equal or higher than 1500 pg/ml a therapy regimen comprising MET pathway inhibitors is recommended. In a more particular embodiment, if the level of sHGF is equal or higher than 1800 pg/ml a therapy regimen comprising MET pathway inhibitors is recommended. More particularly, MET pathway inhibitor therapy regimen is selected/recommended if the level of sHGF is equal or higher than 1886 pg/ml.

On the other hand, SCLC patients with serum HGF levels from 800 pg/ml to 1500 pg/ml correspond to SCLC that will likely not be recommended for receiving a MET pathway inhibition therapy regimen.

In another particular embodiment of the method for selecting a SCLC subject for a therapy regimen comprising MET pathway inhibitors, said therapy regimen further comprises chemotherapeutic agents. Examples of said additional chemotherapeutic agents include topoisomerase inhibitors, such as etoposide and topotecan, as well as platinum-based antineoplastic agents, such as cisplatin and carboplatin. In a more particular embodiment, the therapy regimen comprises combinations of these chemotherapeutic agents in addition to the MET pathway inhibitors.

Combinations of any of these chemotherapeutic agents relates to mixtures in a same composition, or the administration in different compositions of these agents that can be simultaneously or sequentially administered in any order.

Optionally, radiotherapy or any other therapy regimen for cancer may be recommended in conjunction with the MET pathway inhibitors in all those patients with serum or plasma HGF levels higher than the reference control value.

Detection of the levels/amounts of serum or plasma HGF is useful in particular for determining the overall survival (OS) of SCLC in a subject diagnosed of this disease and receiving a therapy regimen. With this aim, the amount or level of serum or plasma hepatocyte growth factor (HGF) in a subject's sample is determined at different times along survival of the subject and a comparison between amounts is done.

In a particular embodiment of this method, it comprises: (a) determining the level of serum or plasma HGF before receiving the therapy regimen; (b) determining the level of serum or plasma HGF measured after receiving the therapy regimen; and (c) comparing these amounts; wherein if the level of (b) is lower than the level of (a), a higher estimated overall survival is determined than if the level of (b) is higher than the level of (a).

Detection of the level of serum or plasma HGF after receiving the therapy regimen corresponds indeed to the response evaluation, which in SCLC subjects receiving therapy is usually taking place 2-3 weeks after the first administration of therapy. Data before starting therapy is also named the baseline, which corresponds to the levels of HGF at diagnosis of the disease by other means.

In a particular embodiment, optionally in combination with any embodiments above or below, the OS is determined in a method wherein: (i) a decrease of the levels of serum or plasma HGF measured for the first time after receiving the therapy regimen (response evaluation) in relation to an amount or levels measured before receiving the therapy regimen (baseline) is indicative of an overall survival from 5.8 months to 13.2 months; and (ii) an increase of the level of serum or plasma HGF measured for the first time after receiving the therapy regimen (response evaluation) in relation to the amount measured before receiving the therapy regimen (baseline) is indicative of an overall survival from 3.5 months to 11 months. In a more particular embodiment: (i) a decrease of the level of serum or plasma HGF measured for the first time after receiving the therapy regimen (response evaluation) in relation to a level measured before receiving the therapy regimen (baseline) is indicative of an estimated overall survival of 9.5 months; and (ii) an increase of the level of serum or plasma HGF measured for the first time after receiving the therapy regimen (response evaluation) in relation to the level measured before receiving the therapy regimen (baseline) is indicative of an estimated overall survival of 7.3 months.

Overall survival may also be determined by comparing the levels of serum or plasma HGF at baseline and at any progression of the disease. Thus, in another particular embodiment of the method for determining the overall survival of SCLC in a subject diagnosed of this disease and receiving a therapy regimen, said method comprises the steps of: (a) determining the level of serum or plasma HGF before receiving the therapy regimen; (b) determining the level of serum or plasma HGF at progression of SCLC; and (c) comparing these levels; wherein if the level of (b) is lower than the level of (a), a higher estimated OS is determined than if the level of (b) is higher than the level of (a).

In a particular embodiment, optionally in combination with any embodiments above or below, the OS is determined in a method wherein: (i) an increase of the level of the serum or plasma HGF measured at progression in relation to a level measured before receiving the therapy regimen is indicative of an overall survival from 6.0 months to 9.9 months; and (ii) a decrease of the level of the serum or plasma HGF measured at progression in relation to the level measured before receiving the therapy regimen is indicative of an overall survival from 7.3 months to 23.0 months. In a more particular embodiment, (i) an increase of the level of the serum or plasma HGF measured at progression in relation to an amount or level measured before receiving the therapy regimen is indicative of an estimated overall survival of 8.0 months; and (ii) a decrease of the level of the serum or plasma HGF measured at progression in relation to the level measured before receiving the therapy regimen is indicative of an estimated overall survival of 15.0 months.

Any of the herewith disclosed in vitro methods, having in common that all give data about the prognosis or outcome of SCLC, may in any particular embodiment or combination of embodiments include a further step of collecting and/or providing and/or saving data derived from previous steps in a data carrier. Thus, the invention also encompasses any data carrier with the prognosis data directly obtained from any of the methods of the invention.

In the sense of the invention a “data carrier” is to be understood as any means that contain meaningful information data for the prognosis of SCLC. Examples of data carrier are printed copies of paper with serum or plasma levels of HGF determined according to these methods and correlating with the prognosis of the disease. The carrier may also be any entity or device capable of carrying the prognosis data. For example, the carrier may comprise a storage medium, such as a ROM, for example a CD ROM or a semiconductor ROM, or a magnetic recording medium, for example a floppy disc or hard disk. Further, the carrier may be a transmissible carrier such as an electrical or optical signal, which may be conveyed via electrical or optical cable or by radio or other means. When the prognosis data are embodied in a signal that may be conveyed directly by a cable or other device or means, the carrier may be constituted by such cable or other device or means. Other carriers relate to USB devices and computer archives.

There exist yet in the market kits and reagents for the analysis of serum or plasma HGF. Examples of these include specific primary monoclonal or polyclonal antibodies recognizing different epitopes of the HGF. These antibodies, together with the appropriate buffers and tools (primary or secondary labelled antibodies, for example) for the detection of the interaction between primary antibodies with HGF are usable for carrying out the method of the invention. Commercial kits include the Quantikine Human Immunoassay (R& D Systems, Minneapolis), HGF Human ELISA Kit, Abcam; HGF Human ELISA Kit (Novex®), Life Technologies; RayBio® Human HGF (SF) ELISA Kit, RayBiotech; Human HGF Instant ELISA, eBioscience; HGF (Human) ELISA Kit, Abnova; Human HGF ELISA Kit, Genway Biotech; and HGF Elisa kit, MyBiosource.

Although immunoassays are particular tests for carrying out the method of the invention, other means, such as nucleic acid analysis technologies may be employed.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

Next examples illustrate how elevated levels of serum HGF in respect of controls are indicative of poor prognosis of SCLC. The controls may include in some cases healthy subjects and in some cases a cohort of SCLC subjects.

For the performance of all the examples, the following patients and methods were selected:

Patients diagnosed with SCLC in Parc de Salut Mar Biobank (MARBiobanc, Barcelona, Spain) Hospital del Mar institution were prospectively included in this study. All participants signed an informed consent. This project was approved by the Local Ethics committee in the institution.

As a control population, there were obtained serum samples from age- and sex-matched healthy donors (N: 30) to the study population.

Serum samples from SCLC patients were obtained at diagnosis before starting any treatment. All patients that were amenable for treatment received standard first line chemotherapy with a combination of platinum (carboplatin or cisplatin) and etoposide at standard doses. Those patients with stage III or less disease received concomitant radiotherapy with radical intent. All patients with responsive disease subsequently received prophylactic cranial irradiation. In this disease, stage III is defined as patients with mediastinal lymph node involvement but no distant metastasis, also called locally advanced (according to the Clinical Cancer Staging protocol of patient classification).

Subsequently, blood samples were obtained at response evaluation (after 3 cycles of chemotherapy for stage IV patients with a metastatic disease and after chemoradiation for stage III or less). Moreover, at first clinically detected progression, blood samples were collected from patients when available. Patients with at least two samples of serum in two different time points were included in the study. All clinical and pathologic data was prospectively included in a specific database. Follow up data was also included with a final cut-off point at November 2013.

Previous observations have showed that serum HGF levels were significantly higher than the plasma levels. Therefore, for a subset of cases plasma samples were also obtained for comparison. Serum and plasma blood samples were collected using serum separator tubes (SST) and anticoagulant (EDTA)-coated tubes, respectively. Samples were allowed to clot for 30 minutes before centrifugation for 10 minutes at 1000 g at 4° C. Serum or plasma was removed and assayed immediately or aliquoted and stored at −20° C.

The Quantikine Human HGF Immunoassay (R&D Systems, Minneapolis, Minn.) was used to measure HGF levels in human serum.

This assay employs the quantitative sandwich immunoassay technique. A monoclonal antibody specific for HGF has been pre-coated onto a microplate. Standards and samples were diluted with the assay diluent, pipetted into the wells and incubated for 2 hours at room temperature. Any HGF present is bound by the immobilized antibody. After washing away any unbound substances, an enzyme-linked polyclonal antibody specific for HGF is added to the wells and incubated for 2 hours at room temperature. Following a wash to remove any unbound antibody-enzyme reagent, a substrate solution is added to the wells and color develops in proportion to the amount of HGF bound in the initial step. The color development is stopped with 2N Sulfuric Acid and the intensity of the color is measured. The optical density of each sample was determined using a microplate reader set at 450 nm. Wavelength correction was set to 540 nm. HGF concentrations were extrapolated from the standard curve generated using the recombinant human HGF of the assay. All samples were run in duplicates.

For the analysis of tumour samples and immunohistochemistry assays in a subset of patients there were analyzed by immunohistochemistry several markers (EMT and p-Met) in the primary tumour to study their association with HGF serum levels. Tumour specimens were retrospectively retrieved from Parc de Salut Mar Biobank (MARBiobanc, Barcelona, Spain). Three μm tissue sections from formalin-fixed and paraffin embedded samples were obtained, mounted onto charged slides and then, deparaffinized in xylene and hydrated.

The following antibodies were used: MET (SP44) mouse mAb (Ventana-Roche, Tucson, Ariz., USA), p-MET Y1234/35 (D26) XP rabbit mAb (Cell Signaling, Danvers, Mass., USA), E-cadherin (NCH-38) mouse mAb (Dako, Carpinteria, Calif., USA), Snail1 (EC3) mouse mAb, and vimentin (V9) mouse mAb (Dako). Immunohistochemistry and in situ hybridization for SPARC protocols have been described elsewhere (see for example in Cañadas et al, supra). Stainings were evaluated by two pathologists independently blinded to clinical information on a light microscope (Olympus DX50, Olympus Corp., Tokyo, Japan). MET, phosphorylated MET (p-MET), and E-cadherin were scored when any percentage of tumour cells was stained in the membrane. Snail1 was evaluated in the nucleus of tumour cells. Vimentin and SPARC were quantified when detected in the cytoplasm of tumour cells. A semiquantitative histoscore (Hscore) was calculated, determined by estimation of the percentage of tumour cells positively stained with low, medium, or high staining intensity for each marker. The final score was determined after applying a weighting factor to each estimate. The formula used was Hscore=(low %)+2×(medium %)+3×(high %), and the results ranged from 0 to 300. Dziadziuszko R, et al., “Correlation between MET gene copy number by silver in situ hybridization and protein expression by immunohistochemistry in non-small cell lung cancer”, Journal of thoracic oncology: official publication of the International Association for the Study of Lung Cancer—2012, Vol. No. 7, pp.: 340-347. The tumours in the present study were classified as p-MET, Snail1, SPARC and vimentin negative when the H-score was 0, vs. positive for any positive H-score. For E-Cadherin and total Met, the median was used as the cut-off for positivity.

The statistical analysis of the data was carried out with the R 3.1 program together with the Statistical Assessment Service from Institut Hospital del Mar d′Investigacions mèdiques (IMIM) in Hospital del Mar (Barcelona).

To analyse associations between categorical variables the Chi-square test or the Fisher's exact test were used, as appropriate. Continuous variables were compared with Mann-Whitney U-test. Spearman correlation coefficient was used to assess correlations between HGF from plasma versus serum. Wilcoxon tests were done to compare sHGF levels from patients at different time points. Overall survival was analysed by Kaplan-Meier method. Curves were compared by the log-rank test. Cox proportional hazards model was used for multivariate analysis. All tests were conducted at the two-sided 0.05 level of significance. This work was performed in accordance with REMARK guidelines, retrievable from McShane et al., “Reporting recommendations for tumour marker prognostic studies”, J Clin Oncol—2005, Vol. No. 23(36), pp.: 9067-72.

### Example 1

### SCLC Patients have Higher HGF Serum Levels when Compared to Healthy Subjects

112 SCLC patients were in this study. SCLC Patients' characteristics are shown in Table 1. As observed, the majority were male, current smokers with good performance status (PS). The metastatic locations were as expected with a majority of patients having liver and bone disease. First line treatment was standard chemotherapy with a higher percentage of patients receiving carboplatin (70%) in combination with etoposide. Patients that were considered unfit for treatment underwent best supportive care. This particular group of patients (N: 9) were characterized by poor PS (2-4) and only had the baseline sHGF sample.

TABLE 1
Patients' characteristics

| N: 112 | | |
|---|---|---|
| Median age (range) | | 66 (29-90) |
| Gender | Male | 89 (79) |
| | Female | 23 (21) |
| Smoking history | Current | 83 (74) |
| | Former | 29 (26) |
| PS- | 0-1 | 77 (69) |
| Performance | | |
| Status^{(a)} | | |
| | 2-4 | 35 (31) |
| Stage^{(a)} | I-III | 30 (27) |
| | IV | 82 (73) |
| Metastatic | Lung | 9 (11) |
| location* | | |
| | Pleura | 18 (22) |
| | Liver | 31 (38) |
| | Bone | 25 (30) |
| | Adrenal | 23 (28) |
| | CNS | 20 (24) |
| Chemotherapy | Cisplatin | 33 (30) |
| | Carboplatin | 69 (62) |
| | None | 9 (8) |

*percentages considering stage IV patients (N: 82)
^{(a)}Disease stage classification is according to Oken et al., “Toxicity And Response Criteria Of The Eastern Cooperative Oncology Group”, Am J Clin Oncol-1982, Vol. 5, pp.: 649-655, 1982.

As differences between serum and plasma levels of HGF have been reported, 26 cases with both type of samples were first analyzed. Correlation between both types of samples with higher levels found in serum, as expected, was determined (data not shown).

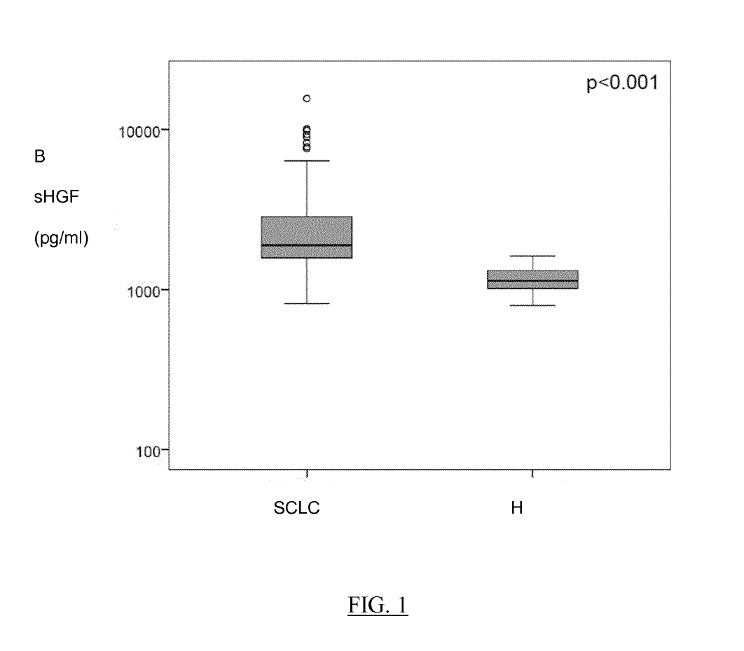

Serum from 30 healthy volunteers matched to the study population by smoking status, gender and age was collected. sHGF levels were variable in healthy subjects ranging from 792 to 1618 pg/ml, with a median sHGF of 1131 pg/ml. sHGF levels for SCLC patients (N:104) at diagnosis were significantly higher than in healthy volunteers with a median of 1886 pg/ml (p<0.001). The range of levels was greater in patients showing values from 816 to 15629 pg/ml. These data are depicted in FIG. 1, wherein in a graphic there is depicted the baseline sHGF levels detected in a cohort of healthy control (H) subjects and in patients with SCLC.

Next, it was evaluated if sHGF was associated with any of the clinical variables. It was found a significant association between higher sHGF and worse (poor) performance status (PS; 2-4) (p=0.002), and presence of liver metastases (p=0.006). This was consistent when it was evaluated sHGF as a continuous variable or dichotomous divided by the median (sHGF=1886 pg/ml).

From these data it is concluded that SCLC subjects have levels/amounts of sHGF higher than a median value defined from a cohort of healthy subjects, wherein healthy subjects are those not suffering from cancer.

### Example 2

### sHGF Levels at Diagnosis and Changes During Treatment have an Impact in Prognosis

It was assessed the impact of all clinical variables on survival in univariate analysis. Median follow up for the series was 7.2 months (0.1-166.4). Table 2 illustrates the association between clinical variables and overall survival (OS). Median overall survival for the whole series was 9.5 months. Increasing age (p=0.002), poor PS, and former smoking history were associated with decreased survival (Table 2). The fact of not receiving treatment was also significantly associated with poorer survival. Within stage IV patients, no differences were observed between patients receiving cisplatin or carboplatin. Regarding location of metastases, those with pleural (p=0.059) or liver metastases (p=0.002) showed decreased survival.

TABLE 2
Univariate analysis between clinical variables and overall survival
(Cox regression model)

| | | | HR (95% CI) | p- |
|---|---|---|---|---|
| | | OS (months) | (hazard ratios) | value |
| Gender | Male | 22.39 (15.56-29.22) | 1.24 (0.68-2.25) | 0.488 |
| | Female | 11.13 (7.75-14.51) | | |
| Tobacco | Current | 23.03 (16.12-29.95) | 2.31 (1.36-3.93) | 0.002 |
| history | | | | |
| | Former | 10.46 (4.46-16.46) | | |
| PS^{(a)} | 0-1 | 24.22 (17.14-31.31) | 3.64 (2.18-6.09) | <0.001 |
| | 2-4 | 9.57 (3.14-15.99) | | |
| Treatment | Yes | 21.64 (15.55-27.72) | 0.061 (0.024-0.15) | <0.001 |
| | No | 1.18 (0.52-1.84) | | |
| Stage^{(a)} | I-III | 40.91 (27.85-53.97) | 4.18 (2.05-8.50) | <0.001 |
| | IV | 11.61 (7.96-15.26) | | |
| Response | Yes | 24.94 (17.77-32.10) | 1.77 (0.75-4.22) | 0.194 |
| to first | | | | |
| line | | | | |
| | No | 11.88 (5.13-18.62) | | |
| sHGF | <1886 | 18.39 (13.49-23.30) | 2.02 (1.21-3.37) | 0.007 |
| (pg/ml) | | | | |
| | >=1886 | 10.45 (6.58-14.32) | | |

^{(a)}Disease stage classification is according to Oken et al., “Toxicity And Response Criteria Of The Eastern Cooperative Oncology Group”, Am J Clin Oncol-1982, Vol. 5, pp.: 649-655, 1982.

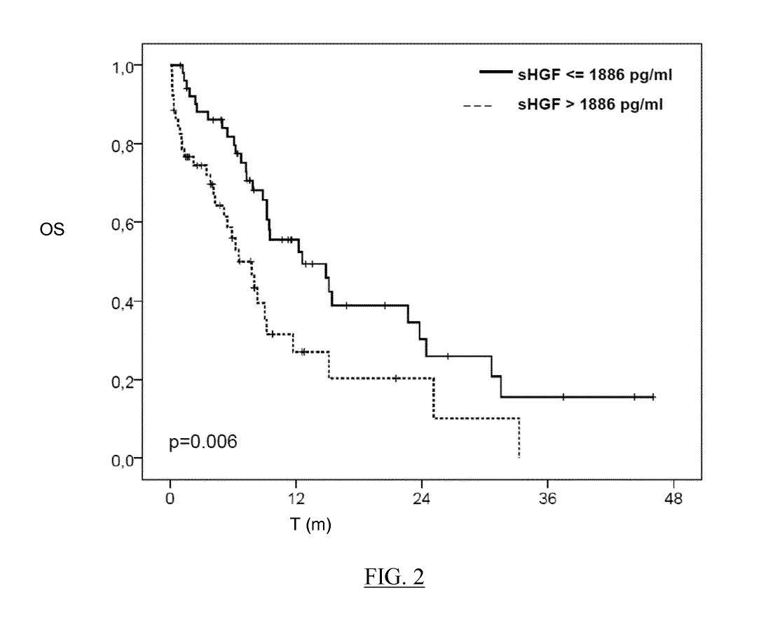

It was next evaluated the impact of sHGF levels at baseline on outcome of these patients. Higher levels of sHGF were associated with worse (poor) survival when analyzed as both continuous or as discrete variable (median of sHGF levels of SCLC as the cut-off). FIG. 2 shows the Kaplan-Meier curve for survival depending on sHGF levels (from Table 2). Moreover, it was determined that increases in 1000 pg/ml were associated with a HR: 1.28 (1.14-1.42) (p<0.001) of dying. This association was also significant when analyzing only stage IV patients (HR: 1.29 (1.14-1.46), p=0.001). It was then performed a multivariate analysis including all significant variables in univariate analysis. Table 3 shows the results of the Cox regression model with the variables that remained independently associated with OS. As observed, higher sHGF levels were associated with poor survival in this model as well as poor performance status PS, former smoking history, advanced stage, the presence of pleural metastases and the lack of treatment.

TABLE 3
Multivariate Cox regression model for OS

| | HR | CI (95%) | p-value |
|---|---|---|---|
| PS^{(a)} (2-4 vs 0-1) | 3.56 | (1.89-6.67) | <0.001 |
| Treatment (Yes vs No) | 0.16 | (0.06-0.44) | <0.001 |
| Stage^{(a)} (IV vs I-III) | 2.98 | (1.31-6.79) | 0.009 |
| Pleural Metastases | 2.05 | (1.01-4.14) | 0.047 |
| (Present) | | | |
| Tobacco (former vs | 1.96 | (1.05-3.67) | 0.035 |
| current) | | | |
| sHGF (>=1886 pg/ml) | 1.94 | (1.13-3.31) | 0.016 |

^{(a)}Disease stage classification is according to Oken et al., “Toxicity And Response Criteria Of The Eastern Cooperative Oncology Group”, Am J Clin Oncol-1982, Vol. 5, pp.: 649-655, 1982.

Briefly, Kaplan Meyer curves or graphics are usable representations showing the survival percentage along time. They provide graphic representation of the gravity of a disease by fixing a value of marker (i.e. a biomarker) and observing how evolve the survival in subjects with a value of the marker over or below the fixed value. This fixed value marker may be a reference control value.

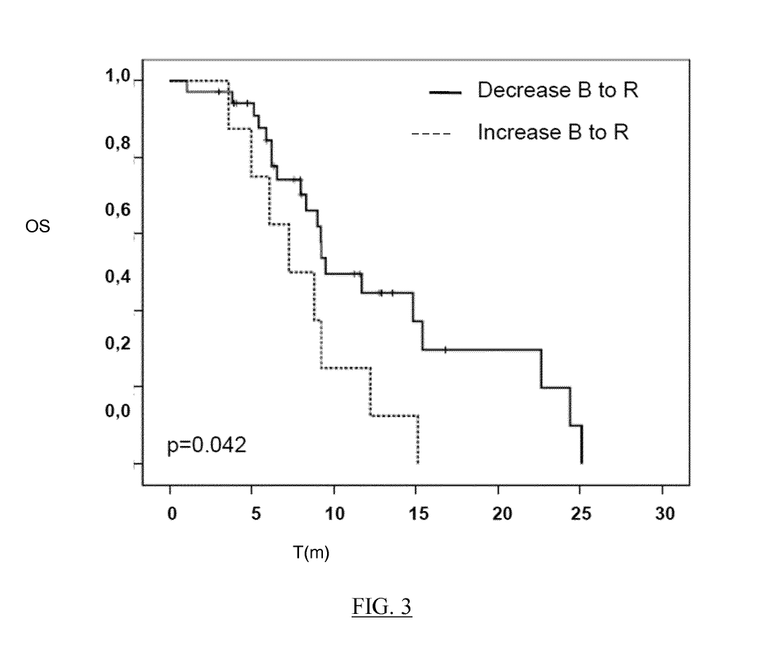

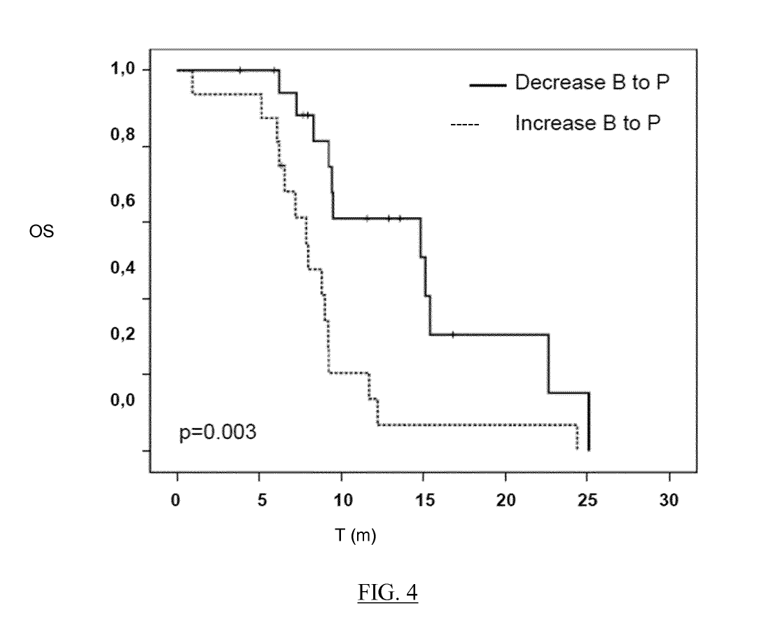

Then it was further evaluated if changes of sHGF during treatment were associated with OS. Stage IV patients were selected because treatment (chemotherapy alone) and outcomes were more homogeneous in this subgroup. Patients who had a decrease of sHGF from baseline to response evaluation presented a longer OS (9.5 months) compared to those that experimented an increase in sHGF at this time point (7.3 months) (p: 0.042) (FIG. 3). Moreover, those patients whose sHGF levels increased from baseline to progression presented shorter survival (8.9 months) vs those whose sHGF was lower at progression (15.2 months) (p: 0.003) (FIG. 4).

Both figures FIGS. 3 and 4 are Kaplan-Meier curves. From them it can be shown that those SCLC patients whose serum levels of HGF decreased at progression or at response in respect of the value at baseline, had a better (high) overall survival (OS).

In order to investigate the potential associations between sHGF levels and response with the impact in survival, there was evaluated the association of sHGF levels and its changes with tumour response. From available information on response to treatment in 89 patients, 6 (7%) patients showed complete response, 75 (84%) partial response, 5 (6%) stable disease and 3 (3%) progression. These categories were not associated with overall survival in a statistically significant manner, although numerically, patients who responded doubled OS compared to those who did not (response to first line p=0.194 in Table 2). No significant correlation was found between response and baseline sHGF levels (median sHGF levels in responders: 1793 pg/ml vs non-responders: 1917 pg/ml). Moreover, sHGF variations during treatment (i.e increase or decrease at response evaluation and progression) were not associated with response either. These observations may be related to the very high percentage of responders in these series.

### Example 3

### Serum HGF Levels Correlate with EMT Phenotype in the Tumour

For a subset of the study population from which enough available tumour samples were available, tumour biomarker analysis was performed. The induction of EMT through MET activation via HGF in SCLC models and the prognostic impact of these markers in human SCLC has been previously reported (see Cañadas et al., supra). The association between sHGF levels and these tumour markers was tested. These markers were assessed in 43 cases, namely for vimentin and Snail1, 44 for SPARC, and p-Met, and 45 for MET and E-cadherin. The percentage of positive cases for each marker is shown in Table 4. Interestingly, it was observed a significant association between increased baseline levels of sHGF (above the median and more frequently over 1886 pg/ml) and Snail1 (p=0.008), vimentin (p=0.038), SPARC (p=0.049) expression and lack of E-cadherin expression (p=0.011). P-MET expression showed a trend towards association with sHGF expression but it did not reach statistical significance (p=0.063) with the cohort of population.

TABLE 4
Biomarker expression in tumour samples

| | | | | | | E- |
|---|---|---|---|---|---|---|
| Biomarker | Met | p-MET | Snail1 | Vimentin | SPARC | cadherin |
| Positive | 22 (49) | 14 (32) | 12 (28) | 10 (23) | 11 (25) | 27 (60) |
| (%) | | | | | | |
| Negative | 23 (51) | 30 (68) | 31 (72) | 33 (77) | 33 (75) | 18 (40) |
| (%) | | | | | | |

These data were consistent with the results of previous works (Cañadas et al, supra) showing around 20-30% of SCLC tumours staining for p-MET, vimentin, Snail1 and SPARC and around 50% of cases considered overexpressed (median as cut-off) for MET and E-cadherin. Thus, sHGF levels in SCLC patients reflected tumor status regarding EMT as evaluated in the biopsy

This means that SCLC patients with sHGF higher than the median of a cohort of SCLC subjects, in particular higher than 1800 pg/ml, and more particularly 1886 pg/ml, will likely respond to a therapy regimen comprising MET pathway inhibitors, thus re-sensitizing to chemotherapy. These SCLC subjects have the advantage that they will be respondent to a chemotherapy regimen to which they would not have been respondent before, due to the mesenchymal phenotype of the tumour cells. Obviously, any chemotherapy regimen may be accompanied by a radiotherapy regimen if so recommended by the oncologist. Data from all the Examples allow affirming that sHGF levels or amounts have an independent role in predicting outcome in patients with SCLC. Patients with higher sHGF show clearly shortened survival and an incremental risk (HR) for death was found with increasing levels of sHGF at the moment of diagnosis by other means. Importantly, sHGF levels are associated with MET pathway activation features in the tumour (mesenchymal phenotype), demonstrating a link between the levels of MET ligand in the serum (i.e. HGF) and the biological effects in the tumour. These results provide novel evidence of the biological relevance of circulating HGF and propose sHGF as a good biomarker for further recommending a therapy regimen with MET pathway inhibitors.

sHGF levels in SCLC patients were able to discriminate patients with poor prognosis upfront. Moreover, changes of serum levels during treatment also predicted for outcome in this disease. The fact that the majority of patients experience a decrease of sHGF levels at response evaluation (where the majority of patients do actually respond to treatment) supports the hypothesis of HGF being secreted at least in part by tumour cells in an autocrine manner, as described in other tumour models.

Globally, these data support the reliability of a serum biomarker to predict for the status of the tumour. And this is of upmost importance for the ability to monitor patients and in particular to potentially select these patients for MET pathway inhibitor therapies.

Results presented here along with other preclinical and clinical data support the evaluation of MET pathway inhibitors in a selected subpopulation of SCLC patients.

### REFERENCES CITED IN THE APPLICATION

Arriola et al, “MET phosphorylation predicts poor outcome in small cell lung carcinoma and its inhibition blocks HGF-induced effects in MET mutant cell lines”, Br J Cancer—2011, Vol—No. 105(6), pp.: 814-23.
Cañadas et al., “Targeting epithelial to mesenchymal transition with Met inhibitors reverts chemoresistance in small cell lung cancer”, Clin Cancer Res—2013.
Toiyama et al., “Serum hepatocyte growth factor as a prognostic marker for stage II or III colorectal cancer patients”, Int J Cancer—2009, Vol. No. 125(7), pp.: 1657-62.
Hosoda et al., “Plasma hepatocyte growth factor elevation may be associated with early metastatic disease in primary lung cancer patients”, Ann Thorac Cardiovasc Surg—2012, Vol. No. 18(1), pp.: 1-7
Ujiie et al“Serum hepatocyte growth factor and interleukin-6 are effective prognostic markers for non-small cell lung cancer”, Anticancer Res—2012, Vol. No. 32(8), pp.: 3251-8.
McShane et al., “Reporting recommendations for tumour marker prognostic studies”, J Clin Oncol—2005, Vol. No. 23(36), pp.: 9067-72.
Dziadziuszko R, et al., “Correlation between MET gene copy number by silver in situ hybridization and protein expression by immunohistochemistry in non-small cell lung cancer”, Journal of thoracic oncology: official publication of the International Association for the Study of Lung Cancer—2012, Vol. No. 7, pp.: 340-347.
Oken et al., “Toxicity And Response Criteria Of The Eastern Cooperative Oncology Group”, Am J Clin Oncol—1982, Vol. 5, pp.: 649-655, 1982.

1. A method for determining the prognosis and treatment of small cell lung cancer (SCLC) in a subject suffering from SCLC, which comprises:
(a) contacting an isolated sample of a subject suffering from SCLC with a reagent selected from an immunoassay reagent or a nucleic acid analysis reagent that binds hepatocyte growing factor (HGF) protein or messenger RNA, wherein the sample is selected from serum and plasma;
(b) measuring the amount of HGF in the isolated sample; and
(c) comparing the amount of HGF in the isolated sample with that of a reference control value; and
(d) determining a prognosis and treatment for the subject,
wherein if the level of HGF is equal or higher than the reference control value, a poor prognosis of SCLC is determined, and the subject is treated with a therapy regimen comprising MET pathway inhibitors.

2. (canceled)

3. The method according to claim 1, wherein the reference control value is the serum or plasma level of HGF resulting from the median of levels of HGF of a cohort of subjects suffering from SCLC.

4. The method according to claim 1, wherein if the level of serum HGF is equal or higher than 1500 pg/ml, a poor prognosis is determined, and the subject is treated.

5. The method according to claim 4, wherein if the level of serum HGF is equal or higher than 1800 pg/ml, a poor prognosis is determined, and the subject is treated.

6. A method for selecting and treating a subject suffering from SCLC, which method comprises:
(a) contacting an isolated sample of a subject suffering from SCLC with a reagent selected from an immunoassay reagent or a nucleic acid analysis reagent that binds hepatocyte growing factor (HGF) protein or messenger RNA, wherein the sample is selected from serum and plasma;
(b) measuring the amount of HGF in the isolated sample; and
(c) comparing the amount of HGF in the isolated sample with that of a reference control value; and
(d) selecting the subject for treatment and treating the subject,
wherein if the level of HGF is equal or higher than a reference control value, the subject is treated with a therapy regimen comprising MET pathway inhibitors.

7. The method according to claim 6, wherein the MET pathway inhibitors are selected from the group consisting of foretinib, crizotinib, onartuzumab, LY2875358, LY2801653, AMG-208, AMG-337, MGCD265, cabozantinib, golvatinib, rilotumumab, flicatuzumab, nintedanib, bevacizumab, dovitinib, danusertib, ponatinib, AZD4547, PD173074, and combinations thereof.

8. (canceled)

9. The method according to claim 6, wherein the reference control value is the serum or plasma level of HGF resulting from the median of levels of HGF of a cohort of subjects suffering from SCLC.

10. The method according to claim 6, wherein if the level of serum HGF is equal or higher than 1500 pg/ml, the subject is treated with a therapy regimen comprising MET pathway inhibitors.

11. The method according to claim 6, wherein if the level of serum HGF is higher than 1800 pg/ml, the subject is treated with a therapy regimen comprising MET pathway inhibitors.

12. The method according to claim 6, wherein the therapy regimen further comprises chemotherapeutic agents selected from the group consisting of topoisomerase inhibitors, platinum-based antineoplastic agents and combinations thereof.

13. The method according to claim 12, wherein the platinum-based antineoplastic agents are selected from cisplatin, carboplatin, and combinations thereof.

14. The method according to any of claim 12, wherein the topoisomerase inhibitors are selected from etoposide, topotecan, and combinations thereof.

15. The method of claim 1 or claim 6, further comprising:
(e) determining the level of hepatocyte growth factor (HGF) in the isolated serum or plasma sample of the subject at different time points and comparing said levels to the reference control value.

16. The method according to claim 15, comprising:
(e1) determining the level of serum or plasma HGF before receiving the therapy regimen;
(e2) determining the level of serum or plasma HGF measured after receiving the therapy regimen; and
(e3) comparing the levels of (e2) and (e1);
wherein if the level of (e2) is lower than the level of (e1), a higher estimated overall survival is determined.

17. The method according to claim 15, comprising:
(e1) determining the level of serum or plasma HGF before start of the therapy regimen;
(e2) determining the level of serum or plasma HGF at progression of SCLC; and
(e3) comparing the levels of (e2) and (e1);
wherein if the level of (e2) is lower than the level of (e1), a higher estimated overall survival is determined.

18. (canceled)

19. (canceled)

20. (canceled)

21. (canceled)

22. (canceled)

23. The method according to claim 1, which further comprises the step of collecting and/or providing and/or saving data derived from previous steps in a data carrier.

24. A method for treating a subject suffering from SCLC, which method comprises
(a) contacting an isolated sample of a subject suffering from SCLC with a reagent selected from an immunoassay reagent or a nucleic acid analysis reagent that binds hepatocyte growing factor (HGF) protein or messenger RNA, wherein the sample is selected from serum and plasma;
(b) measuring the amount of HGF in the isolated sample;
(c) comparing the amount of HGF in the isolated sample with that of a reference control value, and
(d) treating the subject with a therapy regimen comprising MET pathway inhibitors if the level of HGF is equal or higher than the reference control value.

25. The method according to claim 24, wherein the MET pathway inhibitors are selected from the group consisting of foretinib, crizotinib, onartuzumab, LY2875358, LY2801653, AMG-208, AMG-337, MGCD265, cabozantinib, golvatinib, rilotumumab, flicatuzumab, nintedanib, bevacizumab, dovitinib, danusertib, ponatinib, AZD4547, PD173074, and combinations thereof.

26. The method according to claim 24, wherein if the level of HGF is equal or higher than the reference control value, the subject is further treated with a therapy regimen comprising chemotherapeutic agents selected from the group consisting of topoisomerase inhibitors, platinum-based antineoplastic agents and combinations thereof.
